(19) United States
(12) Patent Application Publication
(10) Pub. No.: US 2021/0233028 A1
(43) Pub. Date: Jul. 29, 2021

(54) ACCOMMODATION MEASURE EVALUATION DEVICE, ACCOMMODATION MEASURE EVALUATION PROGRAM, ACCOMMODATION MEASURE EVALUATION METHOD, ACCOMMODATION MEASURE RECOMMENDATION DEVICE, ACCOMMODATION MEASURE RECOMMENDATION PROGRAM, AND ACCOMMODATION MEASURE RECOMMENDATION METHOD

(71) Applicant: IDEPENDENT-URARA LIMITED LIABILITY COMPANY, Chiyoda-ku, Tokyo (JP)

(72) Inventors: Takayuki HONGO, Nagasaka-cho, Hokuto-shi, Yamanashi (JP); Akane SHINOHARA, Chiyoda-ku, Tokyo (JP)

(73) Assignee: IDEPENDENT-URARA LIMITED LIABILITY COMPANY, Chiyoda-ku, Tokyo (JP)

(21) Appl. No.: 17/049,959

(22) PCT Filed: Apr. 22, 2019
(86) PCT No.: PCT/JP2019/018199

§ 371 Date: Oct. 23, 2020

(30) Foreign Application Priority Data
Apr. 23, 2018 (JP) 2018-092575

(51) Int. Cl. G06Q 10/10; G06Q 50/22; G06Q 50/18; G16H 40/00

(52) U.S. Cl. CPC G06Q 10/10; G16H 40/00; G06Q 50/18; G06Q 50/22

(57) ABSTRACT

A device and a method for evaluating what kind of accommodation measure should be taken to reach the reasonable accommodation are provided. This device stores accommodation measure evaluation data that associates an accommodation measure for a minority employee (a person with disabilities, an elderly person, a foreigner) with evaluation of the accommodation measure. When the accommodation measure to be provided for the minority employees is input, the evaluation of the accommodation measure to be provided is acquired based on the input accommodation measure and the accommodation measure evaluation data, and the evaluation is displayed on a display monitor or the like.

### TECHNICAL FIELD

The present disclosure relates to a device and the like for enhancing a reasonable accommodation to persons called social minorities (particularly, minority employees employed by companies or the like) such as persons with disabilities, foreign nationals and elderly persons.

### BACKGROUND

With the adoption and conclusion of the Convention on the Rights of Persons with Disabilities at the United Nations General Assembly and the enforcement of the Act on the Elimination of Discrimination against Persons with Disabilities, entities such as companies are obliged to give a reasonable accommodation to the minority employees. In addition, with the aging of society and globalization, the number of companies employing elderly persons and foreign nationals has been increasing, and it is necessary to give the reasonable accommodation to elderly persons and foreign nationals.

### CITATION LIST

### Patent Literature

Since no mechanism has been proposed so far to evaluate the reasonable accommodation, the prior art documents cannot be specified.

### SUMMARY

### Technical Problem

However, since the minority employees often have special circumstances, employers currently do not know what kind of accommodation is supposed to be given or what kind of accommodation would be the reasonable accommodation.

Therefore, the present disclosure provides the employer with a device and the like for evaluating what kind of accommodation is appropriate and what kind of accommodation measure is supposed to be taken to reach the reasonable accommodation.

### Solution to Problem

In order to solve the above problems, the present disclosure provides a device including the following units, a program that causes a computer to function as the following units, and a method in which the computer performs the following steps.

(1) An accommodation measure evaluation data acquisition unit configured to acquire accommodation measure evaluation data that associates an accommodation measure for a minority employee with an evaluation of the accommodation measure, an accommodation measure acquisition unit configured to acquire the accommodation measure for the minority employee, an evaluation acquisition unit configured to acquire the evaluation of the accommodation measure acquired by the accommodation measure acquisition unit based on the accommodation measure acquired by the accommodation measure acquisition unit and the accommodation measure evaluation data acquired by the accommodation measure data acquisition unit, and an evaluation output unit configured to output the evaluation acquired by the evaluation acquisition unit.

(2) The accommodation measure acquired by the accommodation measure acquisition unit is an accommodation measure that has been provided for the minority employee by an entity that employs the minority employee, or an accommodation measure to be provided for the minority employee by the entity that employs the minority employee, and the evaluation is an evaluation of the accommodation measure that has been provided for the minority employee by the entity that employs the minority employee, or an evaluation of the accommodation measure to be provided for the minority employee by the entity that employs the minority employee.

(3) The minority employee comprises a person with disabilities, a foreign national, or an elderly person.

(4) The evaluation comprises information indicating a relationship with a reasonable accommodation that the entity employing the minority employee should provide. The evaluation comprises information indicating whether criteria for the reasonable accommodation that is supposed to be provided by the entity employing the minority employee are satisfied.

(5) The evaluation comprises information indicating whether criteria for the reasonable accommodation that is supposed to be provided by the entity employing the minority employee are satisfied, and the device comprises a basis generating unit configured to generate a basis indicating validity of having adopted the accommodation measure when the evaluation satisfies the criteria.

(6) The basis comprises a foreign precedent on the accommodation measure, a domestic precedent on the accommodation measure, a case of the accommodation measure published on the Internet, a situation of another entity that adopts the accommodation measure, or the number of other entities adopting the accommodation measure.

(7) The evaluation comprises information indicating whether criteria for the reasonable accommodation that is supposed to be provided by the entity employing the minority employee are satisfied, and the device comprises an unacquired accommodation measure output unit configured to output an unacquired accommodation measure that has not been acquired by the accommodation measure acquisition unit when the evaluation does not satisfy the criteria.

(8) The evaluation comprises data quantifying the evaluation, or data indicating importance of the evaluation.

(9) The device comprises: a first evaluation adjusting unit configured to give a high evaluation when the accommodation measure is a measure desired by the minority employee; a second evaluation adjusting unit configured to give a high evaluation when the accommodation measure is a measure proposed by the entity that employs the minority employee; and a third evaluation adjusting unit configured to give a higher evaluation as the number of entities adopting the accommodation measure increases.

(10) The accommodation measure comprises a measure to protect privacy of the minority employee.

(11) A condition presentation unit configured to present accommodation conditions including the accommodation measure to the minority employee, and an agreement data acquisition unit configured to acquire agreement data indicating that the minority employee agrees to the accommodation conditions.

(12) The accommodation conditions comprise the evaluation.

(13) The accommodation conditions comprise a basis indicating validity of having adopted the accommodation measure.

(14) An accommodation measure selection data acquisition unit configured to acquire accommodation measure selection data that associates an accommodation measure for a minority employee with a state of work of the minority employee, a state acquisition unit configured to acquire the state, a recommended accommodation measure acquisition unit configured to acquire the accommodation measure recommended, based on the state acquired by the state acquisition unit and the accommodation measure selection data acquired by the accommodation measure selection data acquisition unit, and a recommended accommodation measure output unit configured to output the accommodation measure acquired by the recommended accommodation measure acquisition unit.

(15) The accommodation measure selection data is stored by associating the accommodation measure with an evaluation of the accommodation measure.

(16) The state comprises an appearance of the minority employee or a behavior of the minority employee.

(17) The accommodation measure acquired by the accommodation measure acquisition unit is an accommodation measure that matches the state of the minority employee.

### Advantageous Effect

(1) By using the present disclosure, since the evaluation of the accommodation measure to be provided can be known, a person in charge of a company can easily know what kind of accommodation measure is supposed to be adopted, what kind of accommodation measure is supposed to be adopted to be a reasonable accommodation, or the like.

(2) By using the present disclosure, since it is possible to give enough accommodation to the minority employee (person with disabilities, elderly person, foreign national, or the like), it is possible to minimize resignation and trouble of the minority employee.

(3) By using the present disclosure, since it is possible to acquire the evaluation and the basis of the accommodation measure, even if there is a dispute or a trouble over “whether enough accommodation measure has been taken” later, the company can argue that the enough accommodation measure has been taken based on the evaluation and the basis. Further, since the evaluation and the basis are generated based on the precedent and the past case, the company can prove that the enough accommodation measure has been taken based on a legitimate basis. In particular, precedents (foreign precedents) of barrier-free developed countries where barrier-free and accommodation for the minority employee are fulfilled, other than own country, will be a very strong basis in verifying the validity of the accommodation measure adopted by the company.

(4) Reliability of the evaluation and the basis can be further improved by including a situation of the company that adopts the accommodation measure, and the number and ratio of the companies in the evaluation and the basis. Because the fact that many companies adopt the accommodation measure shows that the accommodation measure is considered to be a highly necessary accommodation measure.

(5) If the evaluation is quantified, or ranked according to the importance, the evaluation can be made objective, and the person in charge of the company can easily understand the contents of the evaluation.

(6) If the accommodation measure is highly evaluated when the accommodation measure is desired by the employee, the accommodation measure desired by the employee is likely to be adopted, which makes it easier for the employee to work. Further, if the accommodation measure is highly evaluated when the accommodation measure is a proposal from the company, the company will actively take the accommodation measure, which makes it easier for the employee to work. Furthermore, if the accommodation measure is highly evaluated when the accommodation measure is adopted by many companies, the necessary accommodation measure will be adopted, which makes it easier for the employee to work.

(7) If the accommodation measure is added with a measure to protect the privacy of the employee, protection of the privacy of the employee is promoted, so that the employee can work with peace of mind.

(8) If the accommodation conditions (total of accommodation measures to be provided) are presented to the employee and agreed by the employee, both labor and management can be satisfied and relieved. Further, by obtaining this agreement, even if there is a dispute over “whether enough accommodation measure has been taken” later, the dispute can be resolved promptly and smoothly.

(9) The employee can be satisfied with and agrees to the accommodation conditions by adding the evaluation and the basis to the accommodation conditions.

(10) By using the present disclosure, it is possible to select appropriate accommodation measure based on the state of work (behavior, appearance, or the like) of the employee.

### BRIEF DESCRIPTION OF THE DRAWINGS

FIG. 1 is a schematic diagram of a hardware configuration of the present disclosure.

FIG. 2 is a diagram illustrating one record of an accommodation management file.

FIG. 3 is a diagram illustrating one record of the accommodation management file.

FIG. 4 is a diagram illustrating data on precedents and cases.

FIG. 5 is a diagram illustrating an input screen and data for identifying the type of person with disabilities.

FIG. 6 is a diagram illustrating an input screen of an accommodation measure, data thereof, and a display content of an evaluation and a basis.

FIG. 7 is a diagram illustrating accommodation conditions and contents of agreement data.

FIG. 8 is a diagram illustrating the input screen of accommodation measure, the data thereof, and the display content of the evaluation and the basis.

FIG. 9 is a diagram illustrating the input screen of accommodation measure, the data thereof, and the display content of the evaluation and the basis.

FIG. 10 is a diagram illustrating a screen for identifying a state of work, data thereof, and a display content of a recommended accommodation measure.

FIG. 11 is a diagram illustrating a process for evaluating the accommodation measure.

FIG. 12 is a diagram illustrating a process for recommending the accommodation measure.

### DETAILED DESCRIPTION

An example 1 of the present disclosure will be described with reference to the drawings.

A hardware configuration of this example will be described.

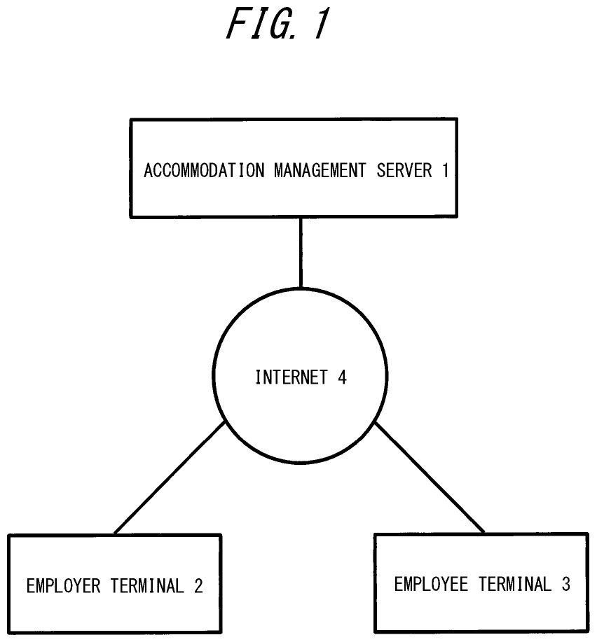

FIG. 1 is a schematic diagram of the hardware configuration of the present disclosure.

The present disclosure includes an accommodation management server 1 (a computer) that performs a process on an accommodation measure for a minority employee, an employer terminal 2 (a computer) used by a person in charge of an entity (a company) that employs the minority employee, and an employee terminal 3 (a computer) used by the minority employee, and devices (the accommodation management server 1, the employer terminal 2, the employee terminal 3) are connected to each other so that they can transmit and receive information via a communication network such as the Internet 4.

Each device stores a computer program (application software) for implementing the present disclosure and predetermined data, and a processing device incorporated in each device performs a predetermined process according to a processing instruction of the computer program.

A structure of the data used in this example will be described.

It is assumed that data items shown in each figure are stored in a storage device in association (correspondence) with each other.

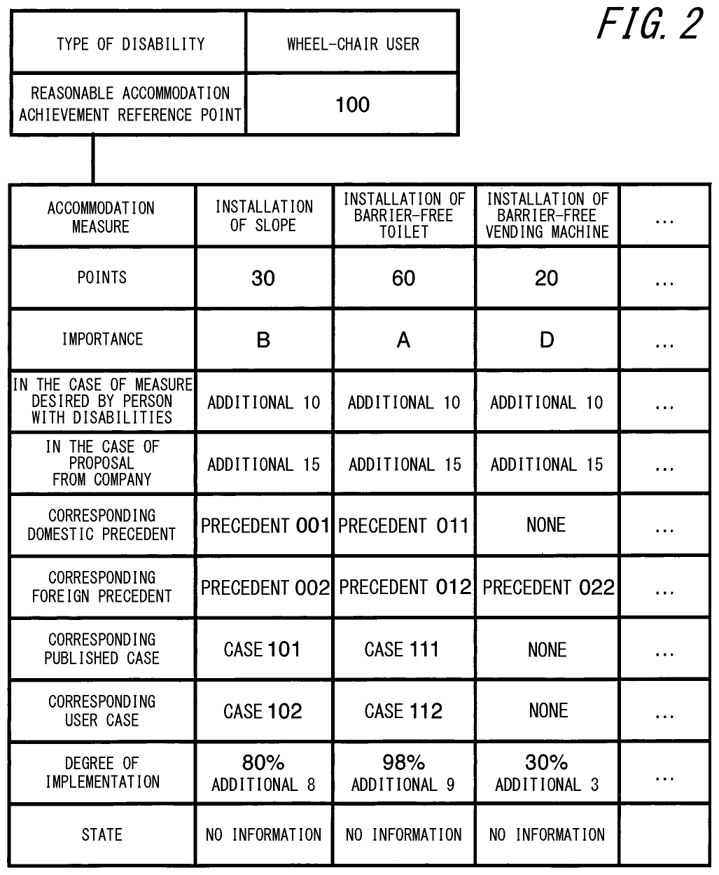

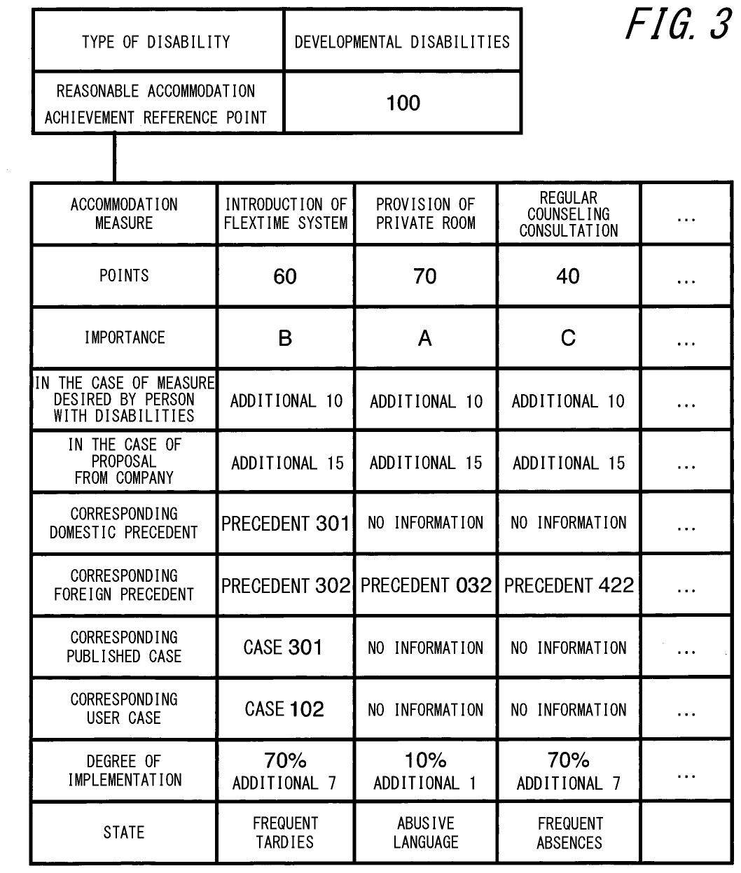

FIGS. 2 and 3 are accommodation management files stored in the accommodation management server 1. FIG. 2 illustrates an accommodation measure for wheelchair users and information thereon, and FIG. 3 illustrates an accommodation measure for persons with developmental disabilities and information thereon. In this example, the accommodation management file serves as accommodation measure evaluation data, and the processing device of the accommodation management server 1 that acquires this from the storage device serves as an accommodation measure evaluation data acquisition unit.

Type of disability means a type of disability, and includes various types of disabilities such as wheelchair users, visual disabilities, hearing disabilities, and developmental disabilities.

Reasonable accommodation achievement reference point is used for determination in which scores (points) given to the accommodation measures adopted by the company are totalized, and if the total score is not less than the reference point, it is determined that “reasonable accommodation is achieved”, and if the total score is less than the reference point, it is determined that “reasonable accommodation is not achieved”.

Accommodation measure is an accommodation measure that the company employing a person with disabilities provides (or plans to provide) for the person with disabilities. Various measures can be considered as the accommodation measures, and they are not limited to those illustrated in this example. The accommodation measures may also include protection of privacy of the employee.

Points are points (data quantifying the evaluation) given to each accommodation measure, and constitutes a part of the evaluation of the accommodation measure. For example, since a barrier-free rest room is an indispensable accommodation measure for the wheelchair users, a high score is given to the accommodation measure. On the other hand, since the wheelchair users will not be much affected even if a barrier-free vending machine or the like is not provided, a high score is not given.

Importance is a data item having the same meaning as the points, and is a rank rather than a score. For example, it is used when it is immediately determined that “reasonable accommodation is not reached” if even one of the highly important accommodation measures such as a rank A is not adopted.

“In the case of measure desired by person with disabilities” is a data item for the purpose of giving a high evaluation (adding points) when the company adopts the measure desired by the person with disabilities (first evaluation adjusting unit). This is to make it easier for the persons with disabilities to fulfill their wishes.

“In the case of proposal from company” is a data item for the purpose of giving a high evaluation (adding points) when the company actively proposes the accommodation measure to the person with disabilities (second evaluation adjusting unit). This is to encourage the company to actively propose the accommodation measure to the person with disabilities.

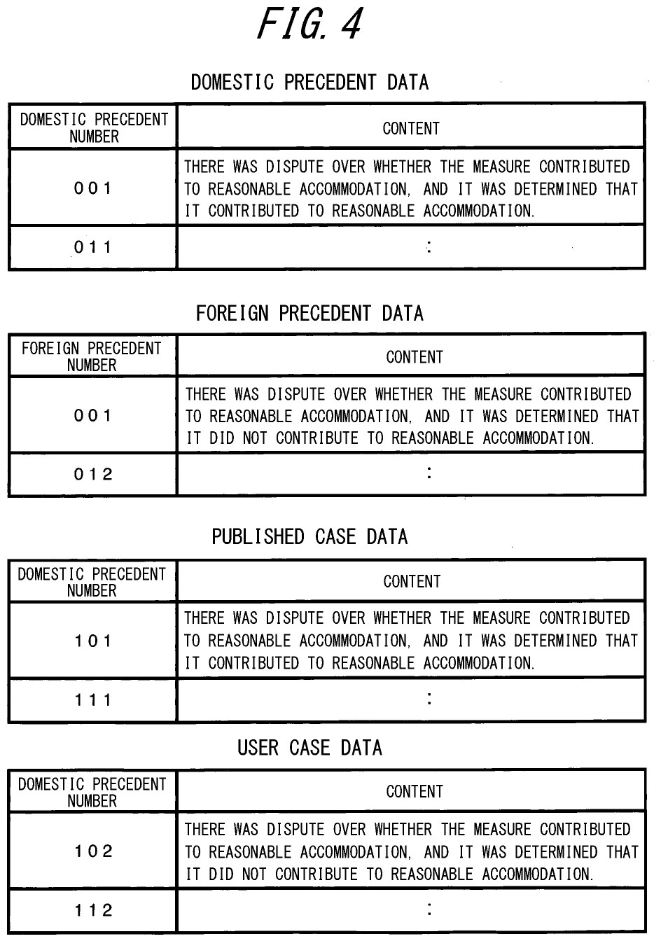

Corresponding domestic precedent means a precedent (precedent number) that held the accommodation measure. By referring to data in FIG. 4 based on this number, the sentences of the court or the summary of judgment can be known. The same applied to corresponding foreign precedent. Corresponding published case is a case published on the Internet or the like (a case having judged the accommodation measure), and corresponding user case is a case indicating data of a case of a person who have used the mechanism of the present disclosure in the past (of a case having judged the accommodation measure).

Degree of implementation indicates the number and ratio of companies that have adopted the accommodation measure (situation of the companies that have adopted the accommodation measure). This data is useful because referring to the number and ratio of the companies will help the person in charge of the company determine whether to adopt the accommodation measure. Further, if the number and ratio of the companies that have adopted the accommodation measure is large, points may be added (third evaluation adjusting unit).

State means an appearance or a behavior of the employed person with disabilities. This data is useful because it is possible to determine what accommodation measures should be adopted based on the state.

A configuration of a display screen used in this example will be described.

The contents displayed on a display monitor and data generated by the input contents are as follows.

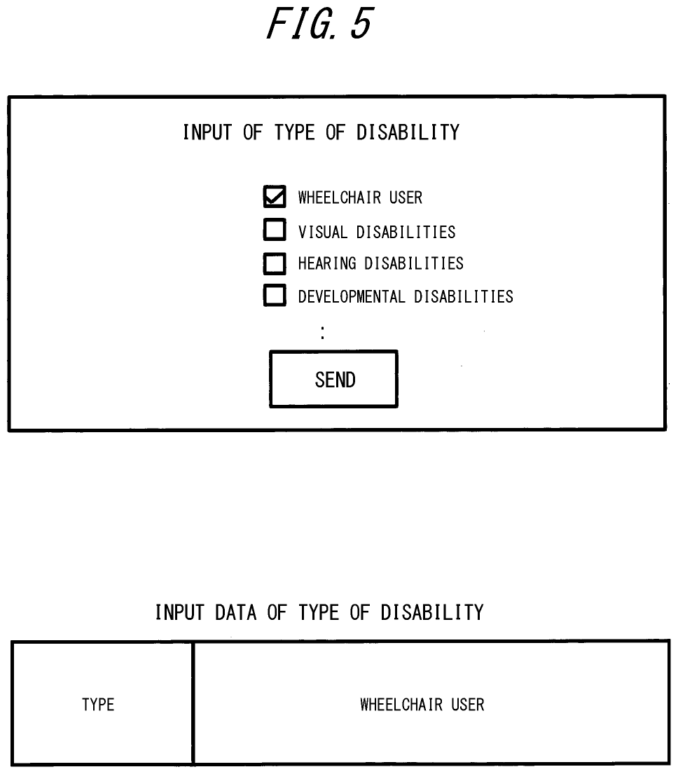

FIG. 5 is a display content for inputting/identifying the type of disability.

In the upper part of FIG. 5, the types of disabilities are listed and displayed, and when the desired type is checked and a send button is clicked (or tapped), input data of the type of disability as in the lower part of FIG. 5 is generated (created).

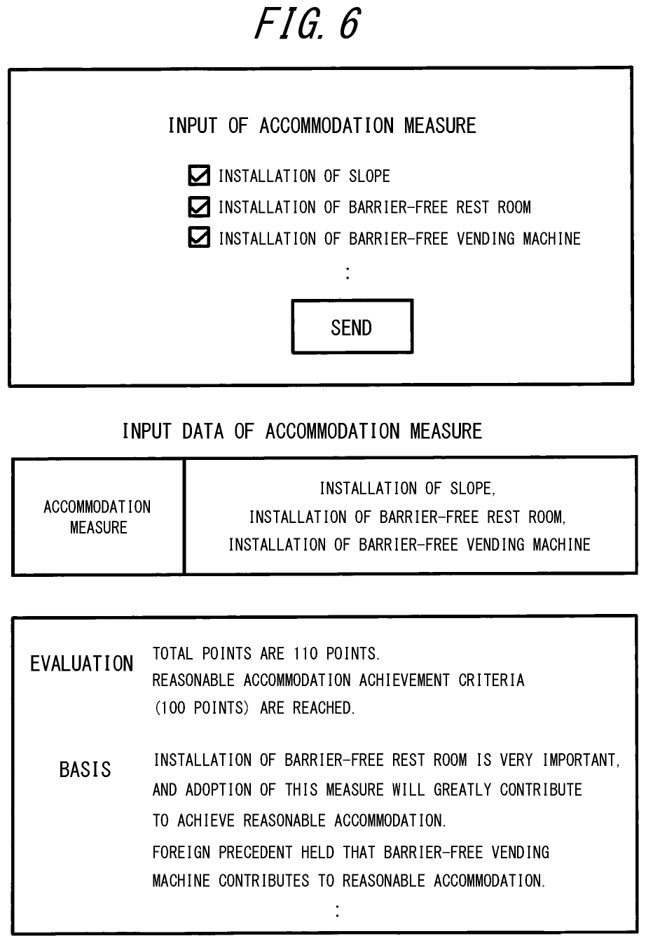

FIG. 6 is a display content for inputting/identifying the accommodation measure that the company provides (or plans to provide).

In the upper part of FIG. 6, the accommodation measures are listed and displayed, and when the accommodation measure to be provided is checked and the send button is clicked, input data of the accommodation measure as in the middle part of FIG. 6 is generated. Thereafter, based on the generated input data of the accommodation measure, a determination whether the accommodation measures have reached the passing score (reasonable accommodation has been reached), and an opinion (a basis) for the accommodation measures is generated and displayed (lower part of FIG. 6).

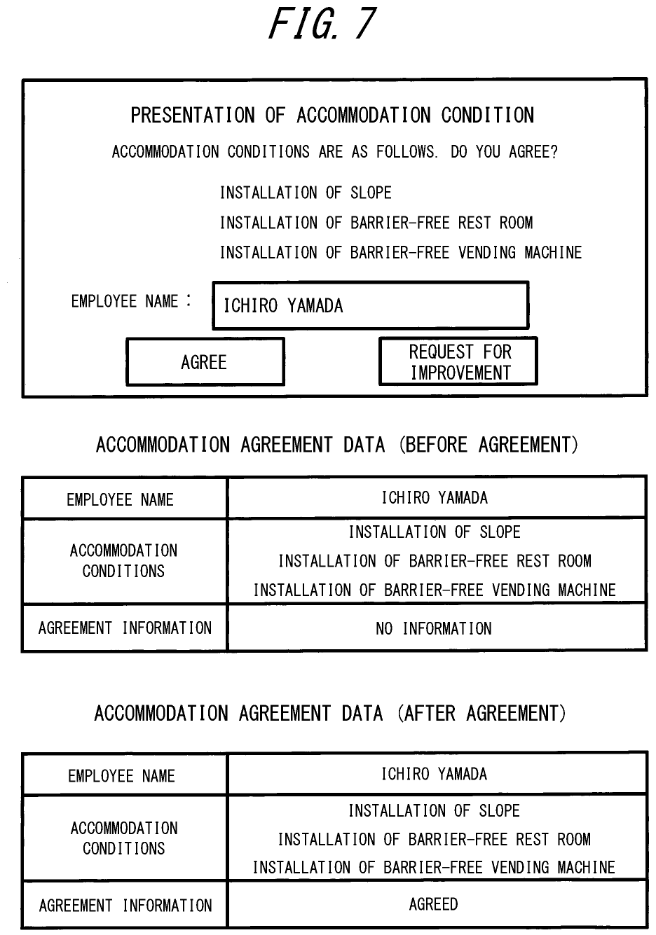

FIG. 7 is a display content in which the accommodation conditions including what kind of accommodation measures the company adopts are presented to the person with disabilities to be employed, and the person with disabilities clearly indicates intention to agree or disagree (request for improvement).

In the upper part of FIG. 7, the accommodation conditions (total of the accommodation measures to be provided) are listed and displayed, and the person with disabilities to be employed inputs the name (employee name) and then clicks an agree button or a request for improvement button. When the agree button is clicked, the accommodation agreement data before agreement as in the middle part of FIG. 6 is updated to the accommodation agreement data after agreement in the lower part of FIG. 6, and it is clear that the employee has agreed to the accommodation measures.

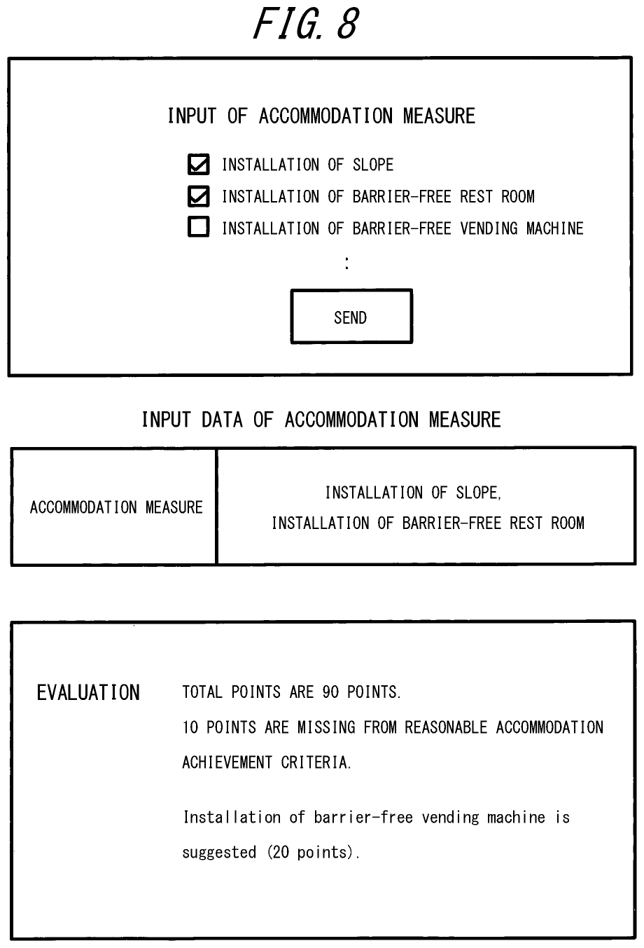

FIG. 8 is the same display content as in FIG. 6 described above. FIG.

8 is an example in which the reasonable accommodation has not been reached, and here, the accommodation measure (installation of the barrier-free vending machine) to be adopted to reach the reasonable accommodation is proposed.

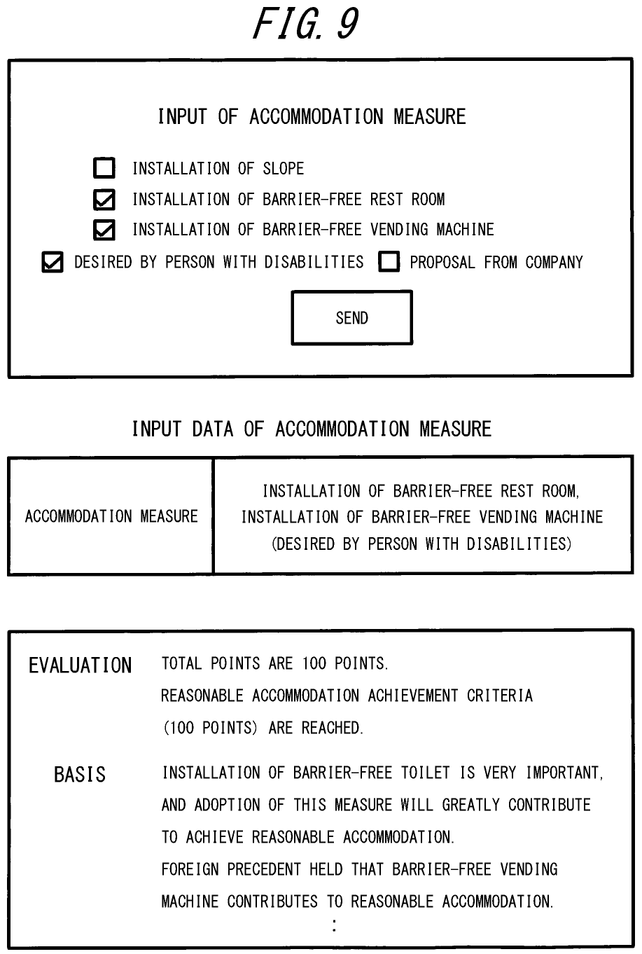

FIG. 9 is the same display content as in FIG. 6 described above. FIG. 9 illustrates a case where the accommodation measure is a measure desired by the person with disabilities.

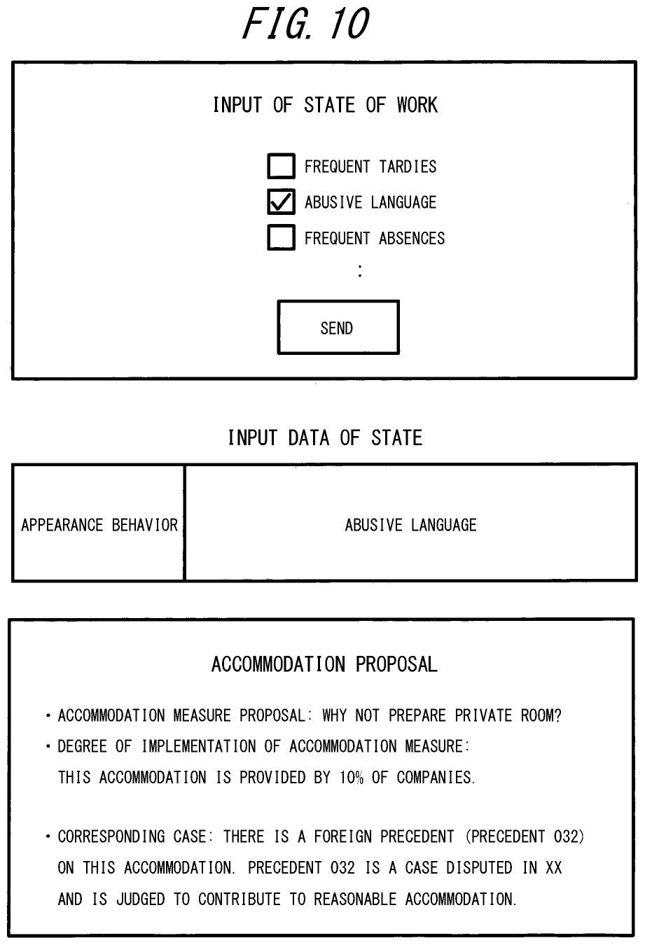

FIG. 10 is a display content for selecting an accommodation measure to be adopted by the company based on the state of the person with disabilities.

In the upper part of FIG. 10, the states (appearance, behavior, or the like) are listed and displayed, and when the applicable state is checked and the send button is clicked, the state input data as in the middle part of FIG. 10 is generated. Thereafter, based on the generated state input data, data on the accommodation measure to be adopted by the company (the accommodation measure that matches the state) is generated and displayed (lower part of FIG. 10).

A process of this example will be described.

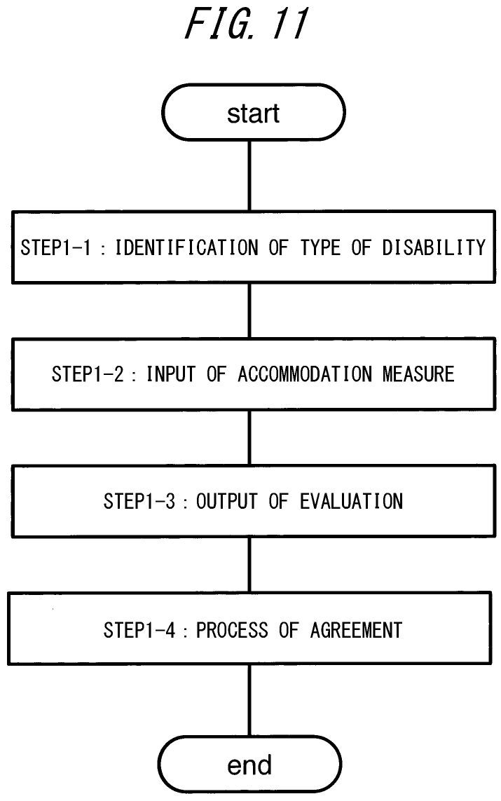

The process is as illustrated in FIG. 11.

STEP1-1 is a step for identifying the type of disability.

It is assumed that a wheelchair user with a lower limb disability newly joined a company.

The person in charge of the company operates the employer terminal 2 to access an input screen for the type of disability in order to determine the accommodation measure for the employee. Thus, the content illustrated in the upper part of FIG. 5 is displayed on the display monitor of the employer terminal 2.

The person in charge selects “wheelchair user” from a list of the types of disabilities displayed as in FIG. 5, and clicks (or taps) the send button. Thus, the employer terminal 2 generates (creates) the input data of the type of disability as in the lower part of FIG. 5 and transmits it to the accommodation management server 1.

The accommodation management server 1 that has received the input data extracts the accommodation measure associated with “type of disability: wheelchair user” from the accommodation management files as in FIGS. 2 and 3 and transmits it to the employer terminal 2.

STEP1-2 is a step for inputting the accommodation measure.

The employer terminal 2 that has received the accommodation measure associated with “type of disability: wheelchair user” from the accommodation management server 1 displays a list of the accommodation measures as in the upper part of FIG. 6. The person in charge checks the accommodation measure to be provided for the wheelchair user who has joined the company and clicks the send button. Thus, the employer terminal 2 generates the input data of the accommodation measure as in the middle part of FIG. 6 and transmits it to the accommodation management server 1.

The accommodation management server 1 (accommodation measure acquisition unit) that has received the input data searches the accommodation management file for the points given to the accommodation measure included in the input data of the accommodation measure. Thus, it is found that installation of the slope is “30 points”, installation of the barrier-free rest room is “60 points”, installation of the barrier-free vending machine is “20 points”, and their total points are “110 points”.

Next, the accommodation management server 1 extracts “reasonable accommodation achievement reference point: 100 points” in FIG. 2 to compare it with the total points “110 points”, and determines that the accommodation measures selected by the person in charge have reached the reasonable accommodation if the total points are not less than the achievement point. On the other hand, if the passing score is less than the achievement point, it is determined that the reasonable accommodation has not been reached. Here, since the total points are not less than the achievement point, the accommodation management server 1 creates the evaluation data (information indicating a relationship with the reasonable accommodation and whether achievement criteria for reasonable accommodation are met) such as “The total points are 110 points. The reasonable accommodation achievement criteria (100 points) have been reached.”

Further, the accommodation management server 1 extracts “corresponding domestic precedent”, “corresponding foreign precedent”, “corresponding public case”, “corresponding user case”, “degree of implementation” (information illustrated in FIG. 2 and FIG. 4) and the like associated with the accommodation measures (installation of the slope, installation of the barrier-free rest room, and installation of the barrier-free rest room) included in the accommodation measure input data, and generates the basis (basis data) illustrated in the lower part of FIG. 6, such as “installation of the barrier-free rest room is very important”, based on the extracted data.

The basis means a basis or appraisal data indicating the validity/rationality of adopting the accommodation measure, and is the data indicating the basis for the evaluation (however, it is not limited thereto).

STEP 1-3 is a step for outputting the evaluation.

Next, the accommodation management server 1 (evaluation output unit) transmits the generated evaluation data and basis data to the employer terminal 2, and the employer terminal 2 having received the data displays it as in the lower part of FIG. 6. Thus, the person in charge understands that the accommodation measures to be provided fulfill reasonable accommodation obligation, and can provide those measures without worries.

STEP1-4 is a step for accommodation measure agreement.

The accommodation management server 1 (accommodation condition presentation unit) transmits the accommodation measures (contents of the input data of the accommodation measures) selected by the person in charge of the company to the employee terminal 3, and the employee terminal 3 that receives the accommodation measures displays a list of accommodation measures as in the upper part of FIG. 7. The accommodation conditions illustrated in FIG. 7 may include the evaluation and the basis. Because the employee can easily determine whether to agree, by reading the evaluation and the basis.

The employee who has read this (newly-employed wheelchair user) inputs his/her name and clicks the “agree button” if he/she agrees with the accommodation conditions, and clicks the “request for improvement” if he/she does not agree. Here, it is assumed that the agree button has been clicked.

The employee terminal 3 transmits application data “employee name: Ichiro Yamada, agreement information: agreed” to the accommodation management server 1, and the accommodation management server 1 (agreement data acquisition unit) that has received the application data updates “agreement information: no information” (middle part of FIG. 7) of the accommodation agreement data stored in the storage device to “agreement information: agreed” (lower part of FIG. 7). This proves that the company and the employee have reached an agreement on the accommodation measures.

Another example of STEP1-2 and STEP1-3 will be described.

FIG. 8 is an example similar to FIG. 6 in principle, but here, unlike the example of FIG. 6, the accommodation measures selected by the person in charge are “installation of slope” and “installation of barrier-free rest room”, and “installation of barrier-free vending machine” has not been selected. In this case, the total points are 90 points, which does not reach the reasonable accommodation achievement reference point of 100 points (needing 10 more points). Therefore, the accommodation management server 1 (unacquired accommodation measure output unit) acquires other accommodation measures (the accommodation measures not acquired by the accommodation measure acquisition unit. Here, installation of barrier-free vending machine.) not selected by the person in charge, and generates the evaluation (evaluation data) in the lower part of FIG. 8, to transmit it to the employer terminal 2. In this way, the person in charge can know that the reasonable accommodation has not been reached and the accommodation measure to be selected in order to reach the reasonable accommodation.

Still another example of STEP1-2 and STEP1-3 will be described.

FIG. 9 is an example similar to FIG. 6 in principle, but here, unlike the example of FIG. 6, as accompanying information of the accommodation measure selected by the person in charge, “whether the accommodation measure is desired by the person with disabilities” and “whether the accommodation measure is positively proposed by the company” are added. For example, in the upper part of FIG. 9, it is possible to input whether “installation of barrier-free vending machine” is “desired by person with disabilities” and whether it is “proposed by company”. When it is “desired by person with disabilities” or “proposed by company”, points indicated in FIGS. 2 and 3 are added to the total points. In this way, wishes by the person with disabilities will be more likely to be adopted, proposals from the company will increase, and the accommodation measures will be enhanced.

Next, an example 2 will be described.

This example selects (recommends) the accommodation measure to be adopted by the company based on the state of work of the employee. The hardware and data used in this example are the same as those in the example 1 in principle.

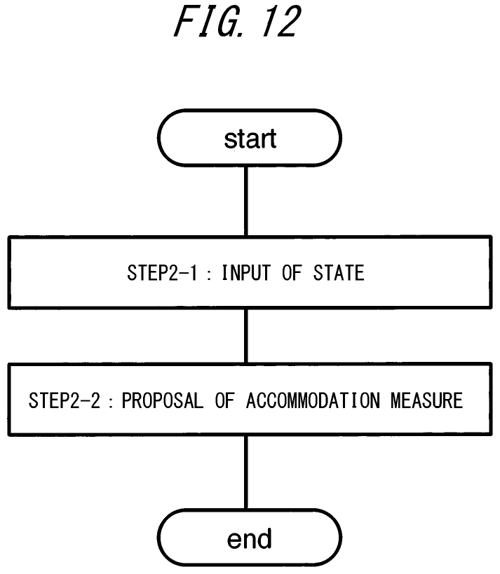

This example is performed according to a process illustrated in FIG. 12. Here, a case of recommending and advising on what kind of accommodation measures should be taken for the employees who often use abusive language during their duties will be described.

STEP 2-1 is a step for inputting the state of work of the employee.

The person in charge of the company accesses the screen of “input state of work” of the employer terminal 2. Thus, the content illustrated in the upper part of FIG. 10 is displayed on the display monitor.

The person in charge checks “abusive language” and clicks the send button. Thus, the employer terminal 2 generates the state input data as in the middle part of FIG. 10, and transmits it to the accommodation management server 1, and the accommodation management server 1 receives it (state acquisition unit).

STEP 2-2 is a step for proposing an appropriate accommodation measure based on the state of work of the employee.

The accommodation management server 1 (recommended accommodation measure acquisition unit) searches the accommodation management file (data for selecting accommodation measures) in FIG. 3 based on “state (appearance/behavior): abusive language” included in the state input data), and extracts the accommodation measure “prepare private room” recommended to the company. Then, the accommodation management server (recommended accommodation measure output unit) generates accommodation proposal data as described in an accommodation proposal in the lower part of FIG. 10 based on this, and transmits the accommodation proposal data to the employer terminal 2.

The employer terminal 2 that has received this displays the accommodation proposal as in the lower part of FIG. 10 on the display monitor.

In this way, the person in charge can take the accommodation measure suitable for the state of work such as behavior and appearance of the employee.

Although a specific case has been described in the above example, the present disclosure is not limited to the above example. For example, other hardware, other data or data items, other processing procedures/processing contents, other display contents, or the like may be used, as long as they can fulfill the same role.

The minority employee is not limited to the person with disabilities. For example, the minority employee may be the foreign national or the elderly person (especially an elderly person of 65 years old or older).

Outputting the data may include transmitting the data, printing the data, displaying the data on the display monitor, or the like. Further, the data transmitted/received between the devices is stored in the storage devices of the devices.

### DESCRIPTION OF REFERENCE NUMERALS AND SIGNS

1: accommodation management server, 2: employer terminal, 3: employee terminal, 4: Internet.

1. An accommodation measure evaluation device comprising:
an accommodation measure evaluation data acquisition unit configured to acquire accommodation measure evaluation data that associates an accommodation measure for a person to be accommodated with an evaluation of the accommodation measure;
an accommodation measure acquisition unit configured to acquire the accommodation measure for the person to be accommodated;
an evaluation acquisition unit configured to acquire the evaluation of the accommodation measure acquired by the accommodation measure acquisition unit based on the accommodation measure acquired by the accommodation measure acquisition unit and the accommodation measure evaluation data acquired by the accommodation measure evaluation data acquisition unit; [[and]] an evaluation output unit configured to output the evaluation acquired by the evaluation acquisition unit;
an unacquired accommodation measure output unit configured to output an unacquired accommodation measure that has not been acquired by the accommodation measure acquisition unit.

2-29. (canceled)

30. The accommodation measure evaluation device according to claim 1, wherein the unacquired accommodation measure output unit presents the accommodation measure to be selected for a person giving accommodation to the person to be accommodated.

31. The accommodation measure evaluation device according to claim 1, wherein
the evaluation comprises information indicating whether criteria for a reasonable accommodation that is supposed to be provided by a person giving accommodation to the person to be accommodated are satisfied, and
the unacquired accommodation measure output unit outputs the unacquired accommodation measure when the evaluation does not satisfy the criteria.

32. The accommodation measure evaluation device according to claim 1, wherein the person to be accommodated comprises a person with disabilities, a foreign national, or an elderly person.

33. The accommodation measure evaluation device according to claim 1, wherein
the person to be accommodated is a minority employee employed by a company or an entity.

34. A non-transitory computer-readable media storing computer-readable instructions that when executed by one or more processors cause the one or more processors to perform operations, the operations comprising;
an accommodation measure evaluation data acquisition step for acquiring accommodation measure evaluation data that associates an accommodation measure for a person to be accommodated with an evaluation of the accommodation measure;
an accommodation measure acquisition step for acquiring the accommodation measure for the person to be accommodated;
an evaluation acquisition step for acquiring the evaluation of the accommodation measure acquired in the accommodation measure acquisition step based on the accommodation measure acquired in the accommodation measure acquisition step and the accommodation measure evaluation data acquired in the accommodation measure evaluation data acquisition step;
an evaluation output step for outputting the evaluation acquired in the evaluation acquisition step; and
an unacquired accommodation measure output step for outputting an unacquired accommodation measure that has not been acquired by the accommodation measure acquisition step.

35. An accommodation measure evaluation method implemented by a computer, the method comprising steps of:
an accommodation measure evaluation data acquisition step for acquiring accommodation measure evaluation data that associates an accommodation measure for a person to be accommodated with an evaluation of the accommodation measure;
an accommodation measure acquisition step for acquiring the accommodation measure for the person to be accommodated;
an evaluation acquisition step for acquiring the evaluation of the accommodation measure acquired in the accommodation measure acquisition step based on the accommodation measure acquired in the accommodation measure acquisition step and the accommodation measure evaluation data acquired in the accommodation measure evaluation data acquisition step;
an evaluation output step for outputting the evaluation acquired in the evaluation acquisition step; and
an unacquired accommodation measure output step for outputting an unacquired accommodation measure that has not been acquired by the accommodation measure acquisition step.

36. An accommodation measure evaluation device comprising:
an accommodation measure evaluation data acquisition unit configured to acquire accommodation measure evaluation data that associates an accommodation measure for a person to be accommodated with an evaluation of the accommodation measure;
an accommodation measure acquisition unit configured to acquire the accommodation measure for the person to be accommodated;
an evaluation acquisition unit configured to acquire the evaluation of the accommodation measure acquired by the accommodation measure acquisition unit based on the accommodation measure acquired by the accommodation measure acquisition unit and the accommodation measure evaluation data acquired by the accommodation measure evaluation data acquisition unit;
an evaluation output unit configured to output the evaluation acquired by the evaluation acquisition unit;
a condition presentation unit configured to present accommodation conditions including the accommodation measure to the person to be accommodated, and
an agreement data acquisition unit configured to acquire agreement data indicating that the person to be accommodated agrees to the accommodation conditions.

37. The accommodation measure evaluation device according to claim 36, wherein the accommodation conditions comprise the evaluation.

38. The accommodation measure evaluation device according to claim 36, wherein the accommodation conditions comprise basis data indicating validity of having adopted the accommodation measure.

39. A non-transitory computer-readable media storing computer-readable instructions that when executed by one or more processors cause the one or more processors to perform operations, the operations comprising;
an accommodation measure evaluation data acquisition step for acquiring accommodation measure evaluation data that associates an accommodation measure for a person to be accommodated with an evaluation of the accommodation measure;
an accommodation measure acquisition step for acquiring the accommodation measure for the person to be accommodated;
an evaluation acquisition step for acquiring the evaluation of the accommodation measure acquired in the accommodation measure acquisition step based on the accommodation measure acquired in the accommodation measure acquisition step and the accommodation measure evaluation data acquired in the accommodation measure evaluation data acquisition step;
an evaluation output step for outputting the evaluation acquired in the evaluation acquisition step;
a condition presentation step configured to present accommodation conditions including the accommodation measure to the person to be accommodated, and
an agreement data acquisition step configured to acquire agreement data indicating that the person to be accommodated agrees to the accommodation conditions.

40. An accommodation measure evaluation method implemented by a computer, the method comprising steps of:
an accommodation measure evaluation data acquisition step for acquiring accommodation measure evaluation data that associates an accommodation measure for a person to be accommodated with an evaluation of the accommodation measure;
an accommodation measure acquisition step for acquiring the accommodation measure for the person to be accommodated;
an evaluation acquisition step for acquiring the evaluation of the accommodation measure acquired in the accommodation measure acquisition step based on the accommodation measure acquired in the accommodation measure acquisition step and the accommodation measure evaluation data acquired in the accommodation measure evaluation data acquisition step;
an evaluation output step for outputting the evaluation acquired in the evaluation acquisition step;
a condition presentation step configured to present accommodation conditions including the accommodation measure to the person to be accommodated, and
an agreement data acquisition step configured to acquire agreement data indicating that the person to be accommodated agrees to the accommodation conditions.
